(19) United States
(12) Patent Application Publication
(10) Pub. No.: US 2007/0260324 A1
(43) Pub. Date: Nov. 8, 2007

(54) Fully or Partially Bioresorbable Orthopedic Implant

(76) Inventors: Ashok V. Joshi, Salt Lake City, UT (US); Akash Akash, Salt Lake City, UT (US)

Correspondence Address: CERAMATEC, INC., 2425 SOUTH 900 WEST, SALT LAKE CITY, UT, 84119, US

(21) Appl. No.: 11/744,560

(22) Filed: May 4, 2007

Related U.S. Application Data
(60) Provisional application No. 60/798,124, filed on May 5, 2006.

(51) Int. Cl. A61F 2/28; A61F 2/44

(52) U.S. Cl. 623/23.51; 623/17.11; 623/23.75

(57) ABSTRACT

A fully or partially bioresorbable orthopedic implant to provide support along a load-bearing axis and a method for producing the same. The implant may include an implant body and a reinforcement material, where the reinforcement material is integrated into the implant body and oriented to provide support along one or more load-bearing axes. The reinforcement material may include a rate of bioresorption that is less than a rate of bioresorption associated with the implant body. In this manner, the fully or partially bioresorbable orthopedic implant of the present invention may facilitate bone ingrowth while providing increased mechanical strength, increased load-bearing capacity, increased bone ingrowth, and decreased propensity for fracture.

This application claims priority to U.S. Provisional Patent No. 60/798,124 filed on May 5, 2006 and entitled REINFORCED, LOAD BEARING PARTIAL-BIORESORBABLE OR FULLY-BIORESORBABLE IMPLANTS AND SCAFFOLDS.

This invention relates to medical implants and more particularly to bioresorbable orthopedic implants for human and animal use.

Orthopedic implants are often used to replace missing, damaged or diseased bone or tissue. A spinal implant, for example, may be implemented to separate and cushion the vertebrae in place of a damaged or diseased intervertebral disc.

Traditionally, spinal and other orthopedic implants have been manufactured of biologically inert materials, such as titanium, tantalum, poly ether ether ketone (“PEEK”), carbon fiber-reinforced plastic (“CFRP”), and bioinert ceramics. Such materials, however, present certain problems in medical applications. Metals such as titanium and tantalum, for example, tend to interfere with x-rays and other imaging techniques. Such metals also have a higher modulus of elasticity than surrounding bone. This disparity in stiffness between the metal implant and surrounding bone may result in a stress shielding effect, where the implant takes the entire load on itself. As a result, the surrounding bone may lose its use and strength over time and cause the implant to loosen. Metal implants are also vulnerable to corrosion, another cause of toxicity concerns in the body.

Other bioinert materials, such as PEEK, and CFRP, may more closely match bone stiffness and avoid interference with medical imaging techniques. Bioinert ceramics also avoid interference with medical imaging techniques. Even these materials, however, pose certain problems in implant applications. Bioinert ceramics, for example, have historically demonstrated low fracture toughness and a propensity for fracture. Also, bioinert metals, plastics, and ceramics may fail to function as a result of the body recognizing the bioinert material as a foreign substance and rejecting it.

Accordingly, recent efforts have been directed to developing bioresorbable implants that are gradually broken down by the body, resorbed, and replaced by advancing tissue, such as bone. Bioresorbable implants are thought to avoid many of the problems typically associated with bioinert implants.

Bioresorbable materials, however, are generally ill-suited to implant applications requiring moderate to high load, shear, or bending stresses. Indeed, most known bioresorbable materials, such as porous calcium phosphate, Bioglass®, poly-L lactic acid (“PLLA”) and polyglycolic acid (“PGA”), are highly porous, with porosity often exceeding fifty percent (50%) of total volume. This high porosity yields low mechanical strength and renders the material prone to fracture. Further, polymer-based implants have poor load-bearing capabilities and often rapidly deteriorate in strength during in-vivo applications.

In view of the foregoing, it would be an improvement to provide a bioresorbable orthopedic implant suitable for load-bearing applications. Beneficially, such bioresorbable orthopedic implants would demonstrate increased mechanical strength and load-bearing capacity and a decreased propensity for fracture. Such a bioresorbable orthopedic implant is disclosed and claimed herein.

The present invention has been developed in response to the present state of the art, and in particular, in response to the problems and needs in the art that have not yet been fully solved by currently available bioresorbable orthopedic implants. In one embodiment in accordance with the invention, an orthopedic implant includes an implant body and a reinforcement material. The implant body includes a first rate of bioresorption. In some embodiments, the implant body includes a material having multiple rates of bioresorption. In other embodiments, the first rate of bioresorption may substantially correspond to a rate of bone ingrowth.

The implant body may include a ceramic and/or a polymer. In some embodiments, for example, the implant body may include tricalcium phosphate (“TCP”), calcium sulfate, calcium carbonate, poly-L lactic acid (“PLLA”), polyglycolic acid (“PGA”), or poly lactic acid (“PLA”). In other embodiments, the implant body may include iodine, iodine compounds, silver, silver compounds, or combinations thereof.

In some embodiments, the implant body includes beads ranging in size between about 0.5 mm and about 3.0 mm. The beads may be round, spherical, cubical, conical, granular, pyramidal, elongated, or hemi-spherical in shape. In one embodiment, the implant body includes pores having diameters ranging between less than about 1 μm to about 700 μm. A porosity of the implant body may range between about greater than zero percent (0%) and about eighty percent (80%) by volume. In some embodiments, the implant body may further include a patterned pore structure yielding decreased propensity for fracture and increased bone ingrowth.

A reinforcement material may demonstrate a rate of bioresorption that is less than the rate of bioresorption associated with the implant body. The reinforcement material may include tricalcium phosphate (“TCP”), calcium sulfate, calcium carbonate, poly-L lactic acid (“PLLA”), polyglycolic acid (“PGA”), or poly lactic acid (“PLA”). In some embodiments, the reinforcement material is bioinert, biocompatible and may include ceramics, metals, and/or plastics. In some embodiments, the reinforcement material may include at least one of alumina, zirconia, silicon carbide, silicon nitride, tantalum carbide, titanium carbide, titanium nitride, titanium oxide, titania, titanium, titanium silicon, tantalum, tantalum carbide, tantalum nitride, tantalum alloys, stainless steel, niobium, niobium alloys, cobalt-chromium alloys, polytetrafluoroethylene, hydroxyapatite, Bioglass®, tri-calcium phosphate (“TCP”), calcium carbonate, calcium sulfate, polyether ether ketone (“PEEK”), carbon fiber reinforced plastic (“CFRP”), polyethylene (“PE”), and ultra high molecular weight polyethylene (“UHMWPE’).

The reinforcement material may include grains, powders, grain boundary constituents, beads, chopped fiber, wires, strands, rod structures, plate structures, cage structures, lattice structures, mesh, and combinations thereof. A cage structure may include a top surface, a bottom surface, and ribs extending between the top and bottom surfaces in a direction substantially corresponding to a load-bearing axis. In other embodiments, the reinforcement material includes a structure oriented in a direction substantially parallel to the load-bearing axis. The reinforcement structure may have a zig zag, a curve, or an annular orientation with respect to the implant body.

The reinforcement material may further include a hollow structure, a porous structure, a substantially solid structure, or combinations thereof. In certain embodiments, the reinforcement material includes a predetermined porosity to substantially match bone stiffness and accommodate bone growth. In certain embodiments, the predetermined porosity may range between about zero percent (0%) and about eighty percent (80%) by volume. Pore sizes may range between about 1 μm and about 700 μm in diameter.

In some embodiments, an end cap may be coupled to the implant body and/or the reinforcement material to accommodate shear forces and help distribute the load across the implant. The end cap may be constructed of alumina, zirconia, silicon carbide, silicon nitride, tantalum carbide, titanium carbide, titania, hydroxyapatite, tri-calcium phosphate (“TCP”), calcium sulfate, calcium carbonate, Bioglass®, titanium, titanium alloys, tantalum, tantalum alloys, stainless steel, niobium, niobium alloys, cobalt-chromium alloys, poly ether ether ketone (“PEEK”), carbon fiber-reinforced plastic (“CFRP”), polyethylene (“PE”) and/or ultra high molecular weight polyethylene (“UHMWPE”).

In some embodiments, a reagent such as an antimicrobial agent, a bactericidal agent, an anti-inflammatory agent, an anti-cancer agent, an anti-infection agent, a pain-relieving agent, a local drug delivery agent, or a bone growth agent may be releasably attached to either or both of the implant body and the reinforcement material.

A method for producing an orthopedic implant to provide support along a load-bearing axis is also presented. In one embodiment, the method includes providing an implant body having a first rate of bioresorption, providing a reinforcement structure having a second rate of bioresorption that is less than the first rate of bioresorption, integrating the reinforcement structure into the implant body, and orienting the reinforcement structure to provide support along the load-bearing axis. Providing an implant body may include integrating a patterned pore structure into the implant body or substantially matching the first rate of bioresorption to a rate of biological material ingrowth.

In some embodiments, a method in accordance with the present invention may also include coupling an end cap to the implant body and/or reinforcement structure. In other embodiments, the method may include releasably coupling a reagent to the implant body and/or reinforcement structure. A reagent may include, for example, an antimicrobial agent, a bactericidal agent, an anti-inflammatory agent, an anti-cancer agent, an anti-infection agent, a pain-relieving agent, a local drug delivery agent, or a bone growth agent.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment, but may refer to every embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

The features and advantages of the present invention will become more fully apparent from the following description and appended claims, or may be learned by the practice of the invention as set forth hereinafter.

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through the use of the accompanying drawings, in which:

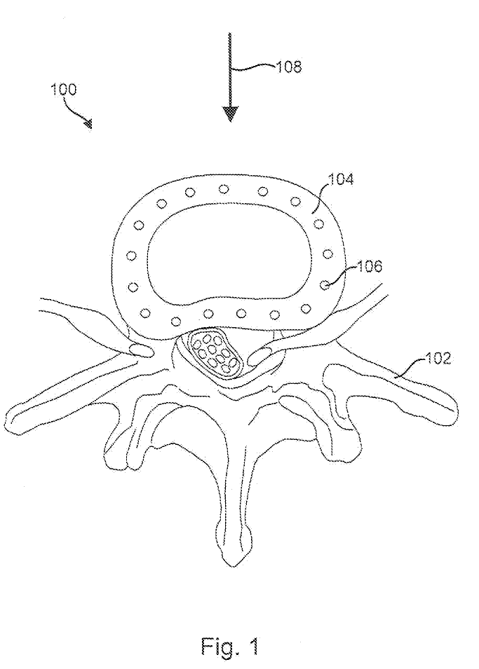

FIG. 1 is a perspective view of one embodiment of a spinal implant characterized by an increased load-bearing capacity in accordance with the present invention;

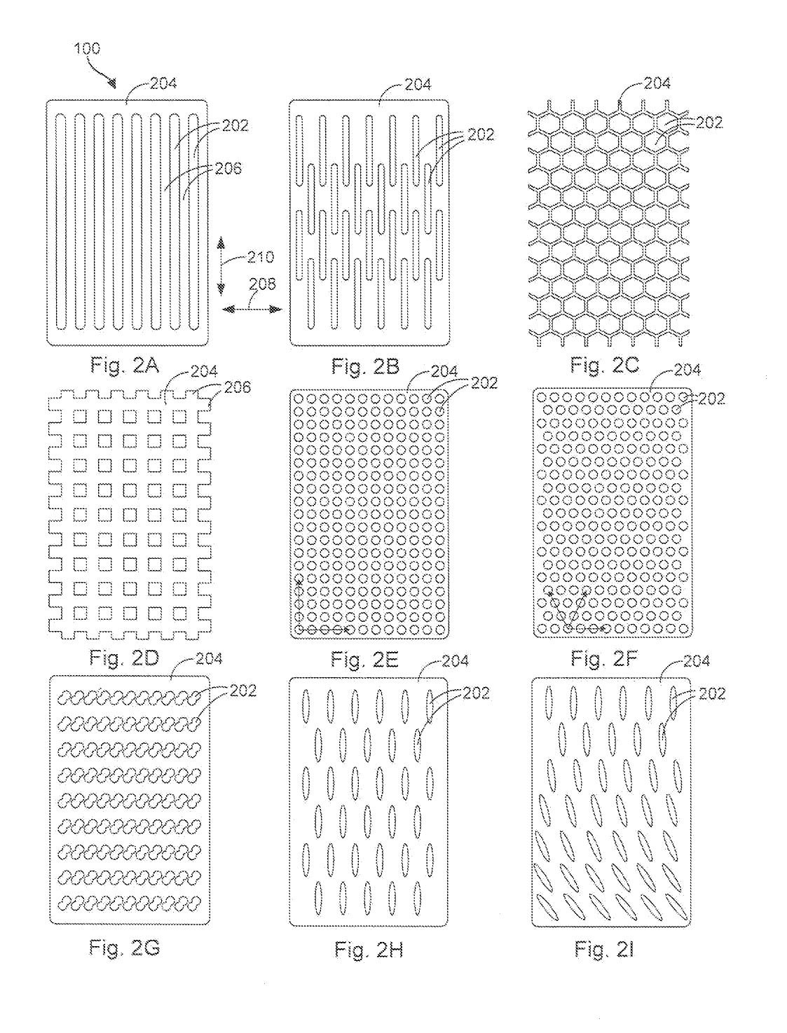

FIGS. 2A-2I are perspective views of various pore structures that may be integrated into an implant body in accordance with certain embodiments of the present invention;

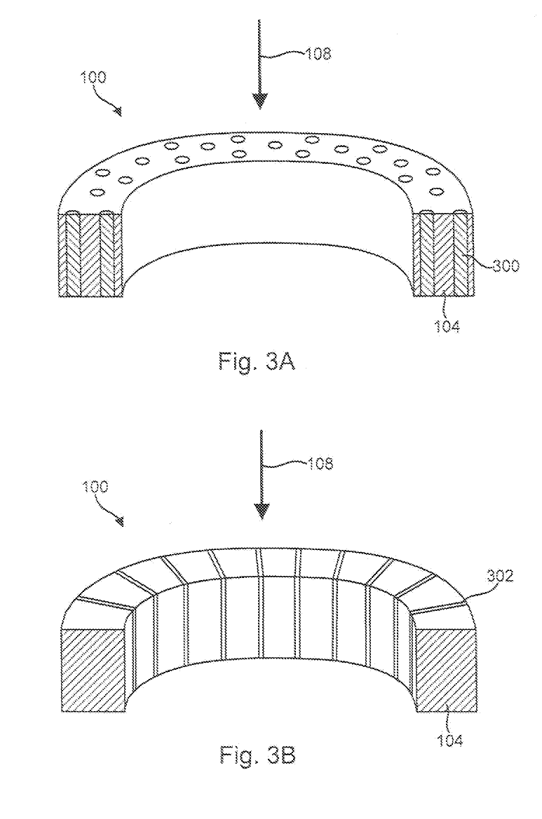

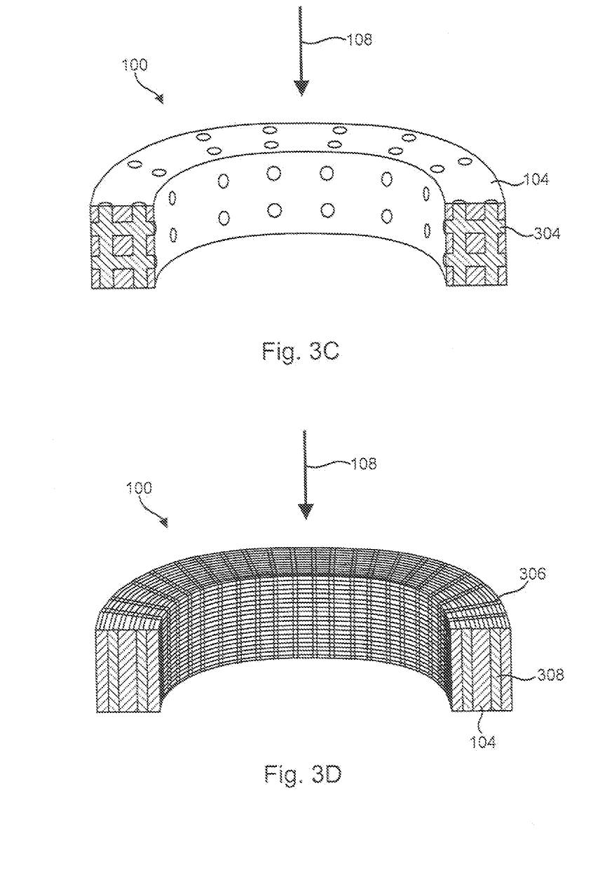

FIGS. 3A-3D are cross-sectional views of a spinal implant showing various reinforcement structures that may be implemented to increase the mechanical strength and/or load-bearing capacity of embodiments of the present invention;

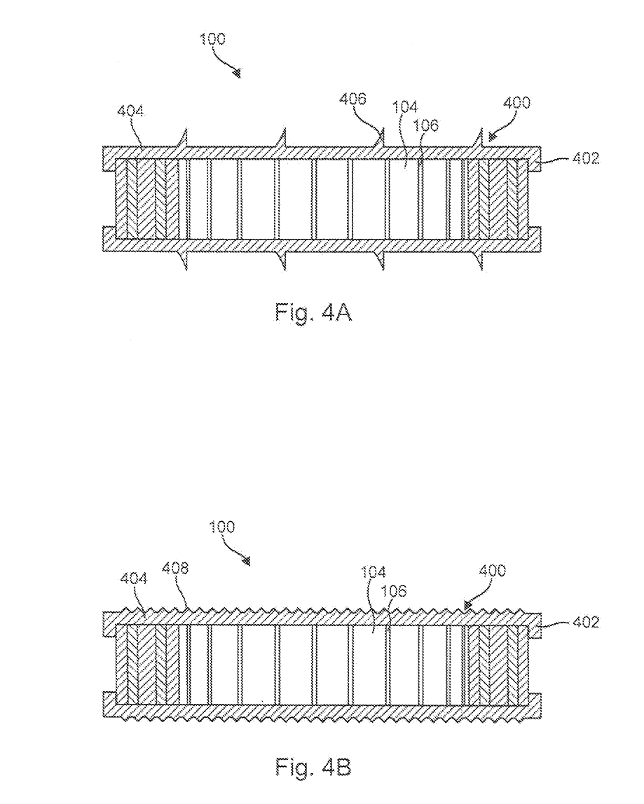

FIGS. 4A and 4B are cross-sectional views of alternative embodiments of orthopedic implants incorporating end caps; and

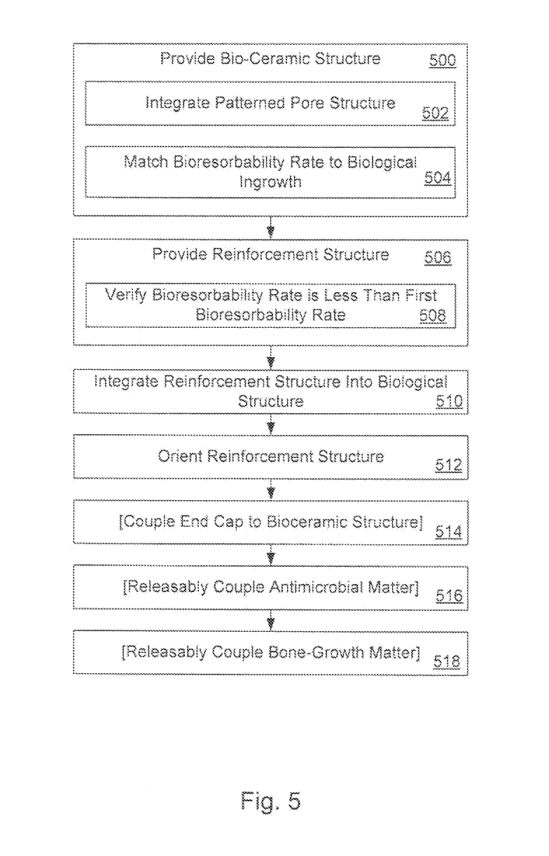

FIG. 5 is a flow diagram of one embodiment of a method for producing an orthopedic implant in accordance with the invention.

It will be readily understood that the components of the present invention, as generally described and illustrated in the Figures herein, could be arranged and designed in a wide variety of different configurations. Thus, the following more detailed description of the embodiments of apparatus in accordance with the present invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

As used herein, the term “ceramic” refers to a chemically inorganic, non-metallic material. The term “bioceramic” refers to a ceramic that is biocompatible, including, for example, alumina, zirconia, calcium phosphates, glass ceramics, pyrolytic carbons, and other such ceramics known to those in the art.

An implant 100 in accordance with the present invention may provide support for orthopedic structures in a physiological environment, as may be required to replace damaged, diseased or missing bone 102 or connective tissue in a human or other animal body. Particularly, an implant 100 may provide orthopedic support along a load-bearing axis. As shown in FIG. 1, for example, an implant 100 in accordance with the present invention may be implemented as a cervical spine implant to cushion cervical vertebrae and provide load-bearing support in the neck area. In other embodiments, the implant 100 may be implemented to provide support in other orthopedic applications known to those in the art.

An implant 100 in accordance with the present invention may comprise any shape and size known to those in the art, and may be particularly formed to resemble the bone or connective tissue it is used to replace. As shown in FIG. 1, for example, the implant 100 may be substantially disc-shaped to resemble a cervical intervertebral disc. The disc may be an open disc having a substantially donut-like shape, or may be substantially solid. One skilled in the art will recognize, however, that an implant 100 in accordance with the present invention is not limited to a disc shape, and may assume any shape and size that is appropriate for its intended application. In some embodiments, corners and edges of the implant 100 may be rounded. In other embodiments, more than one implant 100 may be used in a single orthopedic application. The particular size and shape of the implant 100 may be determined according to the size and shape of the orthopedic defect to be repaired, and may take into account the loading conditions specific to the affected site.

The implant 100 may include an implant body 104 and a reinforcement material or structure 106. The implant body 104 may be composed of one or more biocompatible bioceramics or polymers that are fully or partially bioresorbable. Suitable bioceramics may include, for example, tri-calcium phosphate (“TCP”), calcium sulfate, calcium carbonate, or any other bioresorbable bioceramic material known to those in the art. In some embodiments, the implant body 104 may further include biocompatible, bioresorbable polymers such as poly-L lactic acid (“PLLA”), polyglycolic acid (“PGA”), and poly lactic acid (“PLA”). In other embodiments, the implant body 104 may include iodine, iodine compounds, silver, silver compounds, or combinations thereof.

In certain embodiments, bioceramic materials included in the implant body 104 may be specifically selected to achieve a particular rate of bioresorption in a biological environment. A rate of bioresorption may depend on several factors including, but not limited to, temperature, physical and chemical properties of surrounding biological materials, and the like. In one embodiment, bioceramic materials are selected to reflect a rate of bioresorption that substantially approximates a rate of advancing tissue ingrowth under particular physiological conditions. In another embodiment, bioceramic materials are selected according to their rates of bioresorption at a temperature substantially corresponding to the internal physiological temperature of a human. For example, the bioceramic material selected may have a solubility coefficient and/or solubility product constant of between about 1×10⁻⁴ and about 1×10⁻⁹⁰. In certain embodiments, as discussed in more detail below, the implant body 104 may include a material having multiple rates of bioresorption.

In one embodiment, the implant body 104 may include sintered or loosely held beads (not shown). The beads may assume various shapes, including round, spherical, cubical, conical, granular, pyramidal, elongated, hemi-spherical, or combinations thereof. The beads may range in size between about 0.5 mm and about 3.0 mm, with an aspect ratio ranging between about 1.0 and 10.0. Beads may be packed such that spaces between the beads permit advancing tissue ingrowth. In some embodiments, spaces between the beads may give between about twenty percent (20%) and about eighty percent (80%) porosity by volume. A bioresorption rate of the beads may be controlled by selecting mixtures of component materials, the combination of which demonstrate a desired bioresorption rate.

In selected embodiments, the implant body 104 may include a substantially unitary body having pores to allow for advancing tissue ingrowth. Pores may be in a range of less than about 1 μm to about 700 μm in diameter. Total composition porosity may range between about greater than 0% to about 80% by volume. This configuration may allow for rapid osteointegration into the implant 100. Pores may run continuously through the implant body 104, or may comprise separate voids within or on the surface of the structure 104. Pores may take any form that provides an attachable surface for bone or tissue ingrowth.

As shown in FIGS. 2A-2I, a patterned pore structure 200 may be integrated into the body to decrease its propensity for fracture as well as to promote bone or tissue ingrowth. A patterned pore structure 200 may further modify the implant body's 104 modulus of elasticity to more closely mimic that of natural bone and improve its flexural strength.

An implant 100 having a precisely tailored pore structure 200 may be produced from a layered structure of metal or ceramic green tape 204. Apertures 202 of various shapes and orientations may be cut in these green tapes 204 to create a desired pore structure 200 in the implant 100. FIGS. 2A through 2I show various embodiments of aperture sizes, orientations, and patterns that may be cut in the green tape 204 to produce different pore structures, each of which may be useful in different applications. These patterns do not represent an exhaustive list, but are simply provided to show examples of various pore structures in accordance with the invention.

For example, referring to FIG. 2A, in a selected embodiment, elongated apertures 202 may be cut in a layer of green tape 204 to produce elongated pores in an implant 100. Columns 206 of material may remain between each of the apertures 202. Such a configuration may increase the flexibility of the resulting implant 100 structure in a direction 208 relative to the elongated apertures 202, resulting in a structure with a modified modulus of elasticity. However, the columns 206 may continue to provide substantial support in a direction 40.

Similarly, referring to FIG. 2B, in other embodiments elongated apertures 202 may be provided in a staggered configuration. Such a configuration may provide additional flexibility in a direction 208 while retaining the ability to bear a substantial load in a direction 210. A staggered pattern may also provide improved load-bearing capacity in a direction 208 compared to the pattern shown in FIG. 2A.

Referring to FIG. 2C, in other embodiments, the tape 204 may be cut into a honeycomb structure forming a network of apertures 202 or geometric cells 202. Honeycomb structures are useful in a wide variety of applications due to their high stiffness and low weight. Although of a hexagonal shape in this example, the geometric cells 202 may take on other shapes (e.g., triangles, squares, etc.) as well, although each may have different mechanical properties. In selected embodiments, a honeycomb layer 204 may be sandwiched between less porous layers, such as solid layers, to provide additional rigidity in the plane parallel to the honeycomb layer 204.

Referring to FIG. 2D, in other embodiments, the tape 204 may be cut into a crisscross pattern or other lattice pattern. Such a pattern may be effective to modify an implant's 100 modulus of elasticity while retaining substantial strength and load-bearing capacity along several directions. For example, a crisscross pattern may include columns 206 which are perpendicular to one another. These columns 206 may support significant loads in directions parallel to the columns, providing significant load-bearing capacity in the directions 208, 210. The columns 206 may be oriented, as needed, to support loads from different angles, and do not necessarily need to be oriented perpendicular to one another. Similarly, a crisscross or lattice pattern may include columns 206 that are oriented in more than just two directions.

Referring to FIGS. 2E, 2F, and 2G, in other embodiments, a pattern of circular apertures 202 may be cut in the green tape 204. For example, circular apertures 202 may be arranged in a matrix along two perpendicular axes, as illustrated in FIG. 2E, or along three axes rotated sixty degree relative to one another, as illustrated in FIG. 2F. Implants 100 implementing these patterns may have different mechanical properties. Similarly, in other embodiments, the circular apertures 202 may be formed such that they interconnect, as illustrated in FIG. 2G. Thus, the pore structure of an implant 100 may be designed to include an interconnected network of pores

Referring to FIGS. 2H and 2I, in other embodiments, elongated apertures 202, such as the elliptically shaped apertures 202 shown, may be designed to have a desired directional anisotropy. This anisotropy may be oriented to give an implant 100 various desired mechanical properties, including load-bearing capacity or flexibility in desired directions. This anisotropy may be substantially unidirectional in some cases, as illustrated in FIG. 2H. The orientation of the anisotropy may also vary in the implant 100. As illustrated in FIG. 2I, the anisotropy of the apertures 202 may change based on their location in the implant 100. This may be useful with implants 100 that are curved, subject to varying loads at different locations, or require different mechanical properties at different locations.

Referring now to FIGS. 3A-3D, a reinforcement material or structure 106 may be integrated into the implant body 104 to increase its mechanical strength and load-bearing capacity. The size and shape of the reinforcement structure 106 may vary according to an intended use of the implant 100, as well as depending on the size and shape of its associated implant body 104. The reinforcement structure 106 may be hollow or substantially solid.

The reinforcement material or structure 106 may include biocompatible, bioinert ceramics, metals, and plastics. Suitable reinforcement materials 106 may include, for example, alumina, zirconia, silicon carbide, silicon nitride, tantalum carbide, titanium carbide, titanium nitride, titanium oxide, titania, titanium, titanium silicon, tantalum, tantalum carbide, tantalum nitride, tantalum alloys, stainless steel, niobium, niobium alloys, cobalt-chromium alloys, polytetrafluoroethylene, hydroxyapatite, Bioglass®, tricalcium phosphate (“TCP”), calcium carbonate, calcium sulfate, polyether ether ketone (“PEEK”), carbon fiber reinforced plastic (“CFRP”), polyethylene (“PE”), ultra high molecular weight polyethylene (UHMWPE”), or any other suitable reinforcement material known to those in the art.

In some embodiments, the reinforcement material 106 may be composed of a combination of bioresorbable and non-bioresorbable ceramics, metals, plastics, polymers, and/or any other suitable materials known to those in the art. Such reinforcement materials 106 may be present on a microscopic scale as grains, powders, or grain boundary constituents, or on a macroscopic level as a physical mixture of beads, chopped fiber, wires, strands, mesh, rod structures, plate structures, cage structures, lattice structures, combinations thereof, or other such materials known to those in the art.

In certain embodiments, the reinforcement structure 106 may comprise materials substantially identical to the implant body 104, although, in some embodiments, its density may be substantially more concentrated. The reinforcement structure 106 may include a predetermined level of porosity to substantially match bone or other tissue stiffness and thus avoid a stress shielding effect. Further, the porosity may be specifically determined to accommodate bone ingrowth. In certain embodiments, reinforcement structure 106 porosity may range between about 0% and about 80% by volume, with pore sizes ranging between about 1 μm and about 700 μm.

In some embodiments, the reinforcement structure 106 may comprise materials demonstrating a rate of bioresorption different than the implant body 104 under the same or similar physiological conditions. For example, the reinforcement structure 106 may demonstrate a rate of bioresorption less than the implant body 104 to allow the reinforcement structure 106 to remain in place while the implant body 104 degrades and surrounding bone or other tissue advances and fuses in its place. In one embodiment, for example, the implant body 104 may be made of calcium sulfate with a solubility product constant of about 9.1×10⁻⁶ while the reinforcement structure 106 may be made of silver iodide with a solubility product constant of about 3.0×10⁻¹⁷.

Similarly, in some embodiments, the implant body 104 may have a first rate of bioresorption, while the reinforcement material or structure 106 has a second rate of bioresorption that is less than the first rate of bioresorption. As previously mentioned, the implant body 104 may include materials having multiple rates of bioresorption. In such embodiments, the second rate of bioresorption may be less than the average of the first rates of bioresorption. In other embodiments, the second rate of bioresorption may be less than the slowest of the first rates of bioresorption.

An orthopedic implant 100 in accordance with the present invention may thus be fully bioresorbable, with the reinforcement material 106 degrading at a substantially slower rate than the implant body 104. In this manner, the orthopedic implant 10 may permit bone ingrowth to completely replace the orthopedic implant 100. Alternatively, an orthopedic implant 100 in accordance with the present invention may be partially bioresorbable such that bone ingrowth gradually replaces the implant body 104 portion of the implant 100, while the reinforcement material or structure 106 remains indefinitely.

Judicious selection of component materials having particular qualities and characteristics may effectively determine rates of bioresorption for each of the implant body 104 and reinforcement structure 106. Depending on such component materials, bioresorption may occur over a period of a few days to a period of ten (10) years or more. In some embodiments, the implant body 104 and the reinforcement structure 106 may be made of the same or similar materials, yet demonstrate different rates of bioresorption due to variances in density or other characteristics.

In some embodiments, such as those shown in FIGS. 3A-3C, a reinforcement structure 106 may be mechanically inserted and locked into the implant body 104. Specifically, as shown in FIG. 3A, the reinforcement structure 106 may comprise one or more longitudinal rods 300 oriented within the implant body 104 to substantially parallel one or more load-bearing axes 108. In another embodiment, as shown in FIG. 3B, the reinforcement structure 106 may comprise one or more plates 302 interspersed within the implant body 104 to parallel a load-bearing axis 108. As shown in FIG. 3C, the reinforcement structure 106 may include a lattice structure 304 placed within the implant body 104 to parallel the load-bearing axis 108.

In other embodiments, such as that shown in FIG. 3D, an open cage structure 306 may be injection molded with ribs 308 extending between a top and bottom surface thereof in a direction substantially corresponding to a load-bearing axis 108. The ribs 308 may comprise substantially annular rings, plates, or any other shape known to those in the art. The open cage structure 306 may then be filled with beads or other materials comprising the implant body 104. Openings in the cage structure 306 may be sized to allow for bone ingrowth while effectively retaining the beads or other components that comprise the implant body 104.

Aligning the reinforcement structure 106 substantially parallel to a load-bearing axis 108 may significantly enhance the load-bearing capacity of the composite implant 100. In some embodiments, however, the reinforcement structure 106 may zigzag or curve through the implant body 104, or may form an annular ring. The orientation of the reinforcement structure 106 may facilitate its ability to substantially evenly distribute a load and thereby relieve at least part of the load assumed by the implant body 104. In this manner, embodiments of the present invention may decrease an incidence of implant 100 fracture or breakage during use.

In some embodiments, the reinforcement structure 106 may be further designed to accommodate tensile, shear, compression, and/or other forces known to those in the art. Further, the reinforcement structure 106 may be particularly designed to withstand forces normally expected in the physiological environment for which the implant 10 is intended. For example, an implant 100 designed for implantation as a cervical spine implant in a forty-year-old may include a reinforcement structure 106 particularly designed to withstand forces normally sustained by a healthy forty-year-old cervical intervertebral disc in situ.

Referring now to FIGS. 4A and 4B, a substantially rigid, wear-resistant end cap 400 may be added to the implant 100 to accommodate shear loads, as may be required in spine implant and other applications, and to distribute the load across the implant 100. An end cap 400 in accordance with the present invention may further prevent subsidence or sinking of the implant 100 into less dense cancellous bone or higher density cortical bone.

In some embodiments, the end cap 400 may include a top surface 404 having surface barbs 406 as shown in FIG. 4A, or surface serrations 408 or undulations as shown in FIG. 4B, to provide improved initial stability upon implantation. Such surface barbs 406 or serrations 408 may further facilitate physical interlocking of the implant 100 with adjacent bone, thereby minimizing a possibility of expulsion over time. In alternative embodiments, the top surface 404 may include a roughened texture or other surface characteristics known to those in the art capable of increasing implant 100 stability.

The end cap 400 may be press fit to the implant 100, glued with a biocompatible adhesive, screwed into the implant body 104 or reinforcement material or structure 106, or attached to the implant 100 by any other means known to those in the art. In one embodiment, two end caps 400 may be injection molded into a unitary, open cage-like structure 306, into which the implant body 104 may then be fitted. The end cap 400 may further include a retaining lip 402 extending from the top surface 404 to further secure the end cap 400 to the implant 100.

The end cap 400 may include biocompatible ceramic, metal, plastic, or any other suitable biocompatible material known to those in the art. In some embodiments, the end cap 400 may be fully or partially bioresorbable. Alternatively, the end cap 400 may be bioinert. Ceramic end caps 400 may include one or more mixtures of alumina, zirconia, silicon carbide, silicon nitride, tantalum carbide, titanium carbide, titania, hydroxyapatite, tri-calcium phosphate (“TCP”), calcium sulfate, calcium carbonate, Bioglass®, or any other suitable ceramic known to those in the art. Metal end caps 400 may include one or more mixtures of titanium, titanium alloys, tantalum, tantalum alloys, stainless steel, niobium, niobium alloys, cobalt-chromium alloys, or any other suitable metal known to those in the art. Plastic end caps 400 may include PEEK, CFRP, or any other suitable plastic known to those in the art.

The end cap 400 may include a level of porosity predetermined to enable stiffness matching with surrounding bone and tissue and to facilitate bone ingrowth and bony fusion. In some embodiments, the end cap 400 may have a porosity range between about zero percent (0%) and eighty percent (80%) by volume, with pore sizes ranging between about less than 1 μm to about 700 μm.

In certain embodiments, pores in the end cap 400, implant body 104, and/or reinforcement material or structure 106 may be impregnated with bone growth factors to encourage rapid healing and complete bone ingrowth upon implantation of the implant 100. Bone growth factors may include, for example, bone morphogenic proteins, osteoconducting elements and compounds, collagen fibers, blood cells, osteoblast cells, and/or other suitable constituents known to those of ordinary skill in the art.

In other embodiments, end cap 400 pores, implant body 104 pores, and/or reinforcement material or structure 106 pores may be further impregnated with or contain health reagents such as antibiotic drugs, anti-inflammatory drugs, cancer drugs, anti-infection drugs, pain-relieving drugs for localized drug delivery and controlled drug delivery, and the like. Health reagents may further include biocompatible silver, halides, halogens, peroxides, and compounds and mixtures thereof. In one embodiment, for example, the health reagent includes one of calcium peroxide, magnesium peroxide, or silver peroxide.

In some embodiments, the health reagents and/or growth factors may be mixed with component materials used to form the implant body 104 or reinforcement material or structure 106, such that the health agents and growth factors are integrated into the implant body 104 or reinforcement material 106 itself. Alternatively, the health reagents and/or growth factors may be attached to either or both of the implant body 104 and reinforcement material or structure 106 by hot pressing, adhesion, pressure, immersion, or any other means of attachment known to those in the art. In any case, the health reagent and/or growth factor may constitute between about one-tenth of one percent (0.1%) and about ten percent (10%) by implant 100 volume, either individually or collectively.

In some embodiments, the means of attachment used to attach the health reagent and/or growth factor to the implant body 104 or reinforcement material or structure 106 may contribute to a desired rate of release of such reagents. For example, in one embodiment, iodine is integrated into the composition of the implant body 104 so it may be slowly released as the implant body 104 resorbs into the biological environment.

Referring now to FIG. 5, a method for producing a bioresorbable orthopedic implant 100 in accordance with embodiments of the present invention may include providing 500 an implant body, providing 506 a reinforcement structure, integrating 510 the reinforcement structure into the implant body, and orienting 512 the reinforcement structure to provide additional support along a load-bearing axis.

Providing 500 an implant body may include integrating 502 within the implant body a patterned pore structure, as discussed in detail with reference to FIGS. 2A-2I above. Providing 500 an implant body may further include substantially matching 504 a rate of bioresorption of the implant body to a rate of bone ingrowth under certain physiological conditions.

Providing 506 a reinforcement structure may include verifying 508 that a rate of bioresorption of the reinforcement structure is less than the rate of bioresorption of the implant body. Providing varying rates of bioresorption in this manner may permit the reinforcement structure to continue to provide at least partial load-bearing support while advancing tissue ingrowth gradually replaces the implant body.

In certain embodiments, a method in accordance with the present invention may further include coupling 514 an end cap to the implant body to provide increased resilience against shear forces, as discussed in detail with reference to FIGS. 4A and 4B above. In other embodiments, the method may further include releasably coupling 516,518 to either of the implant body or the reinforcement structure antimicrobial matter or bone growth matter, also discussed above with reference to FIGS. 4A and 4B.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An orthopedic implant to provide support along a load-bearing axis, the implant: comprising:
a biocompatible implant body having a first rate of bioresorption; and
a reinforcement material integrated into the implant body and oriented to provide support along the load-bearing axis, the reinforcement material having a second rate of bioresorption that is less than the first rate of bioresorption.

2. The implant of claim 1, wherein the implant body comprises a patterned pore structure.

3. The implant of claim 1, wherein the implant body further comprises a material having multiple rates of bioresorption.

4. The implant of claim 1, wherein the implant body comprises at least one material chosen from a ceramic and a polymer.

5. The implant of claim 1, wherein the implant body comprises a material chosen from tricalcium phosphate (“TCP”), calcium sulfate, calcium carbonate, poly-L lactic acid (“PLLA”), polyglycolic acid (“PGA”), and poly lactic acid (“PLA”).

6. The implant of claim 1, wherein the bioceramic implant body comprises at least one material chosen from iodine, iodine compounds, silver, silver compounds, and combinations thereof.

7. The implant of claim 1, wherein the reinforcement material comprises a material chosen from tricalcium phosphate (“TCP”), calcium sulfate, calcium carbonate, poly-L lactic acid (“PLLA”), polyglycolic acid (“PGA”), and poly lactic acid (“PLA”).

8. The implant of claim 1, wherein the reinforcement material is bioinert and biocompatible and comprises at least one material chosen from ceramics, metals and plastics.

9. The implant of claim 1, wherein the reinforcement material comprises material chosen from alumina, zirconia, silicon carbide, silicon nitride, tantalum carbide, titanium carbide, titanium nitride, titanium oxide, titania, titanium, titanium silicon, tantalum, tantalum carbide, tantalum nitride, tantalum alloys, stainless steel, niobium, niobium alloys, cobalt-chromium alloys, polytetrafluoroethylene, hydroxyapatite, Bioglass®, tricalcium phosphate (“TCP”), calcium carbonate, calcium sulfate, polyether ether ketone (“PEEK”), carbon fiber reinforced plastic (“CFRP”), polyethylene (“PE”), and ultra high molecular weight polyethylene (“UHMWPE”).

10. The implant of claim 1, wherein the reinforcement material comprises a form chosen from grains, powders, grain boundary constituents, beads, chopped fiber, wires, strands, rod structures, plate structures, cage structures, lattice structures, mesh, and combinations thereof.

11. The implant of claim 10, wherein the cage structure comprises a top surface, a bottom surface, and ribs extending between the top and bottom surfaces in a direction substantially corresponding to the load-bearing axis.

12. The implant of claim 10, wherein the reinforcement material comprises a structure oriented in a direction substantially parallel to the load-bearing axis.

13. The implant of claim 10, wherein the reinforcement material comprises a structure chosen from a zig zag, a curve, and an annular orientation with respect to the implant body.

14. The implant of claim 1, wherein the reinforcement material comprises a structure chosen from a hollow structure, a porous structure, a substantially solid structure, and combinations thereof.

15. The implant of claim 14, wherein the reinforcement material comprises a predetermined porosity to substantially match bone stiffness and accommodate bone ingrowth.

16. The implant of claim 14, wherein the predetermined porosity comprises between about zero percent (0%) and about eighty percent (80%) by volume.

17. The implant of claim 14, wherein the reinforcement material comprises pores ranging between about 1 μm and about 700 μm in diameter.

18. The implant of claim 1, wherein the implant body comprises beads ranging in size between about 0.5 mm and about 3.0 mm.

19. The implant of claim 18, wherein the beads comprise at least one of a round, spherical, cubical, conical, granular, pyramidal, elongated and hemi-spherical shape.

20. The implant of claim 1, wherein the implant body comprises pores having diameters ranging between less than about 1 μm to about 700 μm.

21. The implant of claim 1, wherein the implant body comprises a porosity of between about greater than zero percent (0%) and about eighty percent (80%) of the implant body by volume.

22. The implant of claim 1, wherein the first rate of bioresorption substantially corresponds to a rate of biological material ingrowth.

23. The implant of claim 1, further comprising an end cap coupled to at least one of the implant body and the reinforcement material.

24. The implant of claim 23, wherein the end cap comprises at least one material chosen from alumina, zirconia, silicon carbide, silicon nitride, tantalum carbide, titanium carbide, titania, hydroxyapatite, tri-calcium phosphate (“TCP”), calcium sulfate, calcium carbonate, Bioglass®, titanium, titanium alloys, tantalum, tantalum alloys, stainless steel, niobium, niobium alloys, cobalt-chromium alloys, PEEK, CFRP, PE, and UHMWPE.

25. The implant of claim 1, further comprising a reagent releasably attached to at least one of the implant body and the reinforcement material.

26. The implant of claim 25, wherein the reagent comprises at least one agent chosen from an antimicrobial agent, a bactericidal agent, an anti-inflammatory agent, an anti-cancer agent, an anti-infection agent, a pain-relieving agent, a local drug delivery agent, and a bone growth agent.

27. A method for producing an orthopedic implant to provide support along a load-bearing axis, the method comprising:
providing an implant body having a first rate of bioresorption;
providing a reinforcement structure having a second rate of bioresorption, wherein the second rate of bioresorption is less than the first rate of bioresorption;
integrating the reinforcement structure into the implant body; and
orienting the reinforcement structure to provide additional support along the load-bearing axis.

28. The method of claim 27, wherein providing an implant body further comprises integrating a patterned pore structure into the implant body.

29. The method of claim 27, wherein providing an implant body further comprises substantially matching the first rate of bioresorption to a rate of biological material ingrowth.

30. The method of claim 27, further comprising coupling an end cap to at least one of the implant body and the reinforcement structure.

31. The method of claim 27, further comprising releasably coupling a reagent to at least one of the implant body and the reinforcement structure.

32. The implant of claim 31, wherein the reagent comprises at least one agent chosen from an antimicrobial agent, a bactericidal agent, an anti-inflammatory agent, an anti-cancer agent, an anti-infection agent, a pain-relieving agent, a local drug delivery agent, and a bone growth agent.

33. An orthopedic implant produced by the steps of:
providing an implant body having a first rate of bioresorption;
providing a reinforcement structure having a second rate of bioresorption, wherein the second rate of bioresorption is less than the first rate of bioresorption;
integrating the reinforcement structure into the implant body; and
orienting the reinforcement structure to provide support along the load-bearing axis.

34. The implant of claim 33, wherein providing an implant body further comprises integrating a patterned pore structure into the implant body.

35. The implant of claim 33, wherein providing an implant body further comprises substantially matching the first rate of bioresorption to a rate of biological material ingrowth.

36. The implant of claim 33, further comprising coupling an end cap to at least one of the implant body and the reinforcement structure.

37. The implant of claim 36, further comprising releasably coupling a reagent to at least one of the implant body, the end cap, and the reinforcement structure.

38. The implant of claim 37, wherein the reagent comprises at least one agent chosen from an antimicrobial agent, a bactericidal agent, an anti-inflammatory agent, an anti-cancer agent, an anti-infection agent, a pain-relieving agent, a local drug delivery agent, and a bone growth agent.
